(19) United States
(12) Patent Application Publication
(10) Pub. No.: US 2025/0120637 A1
(43) Pub. Date: Apr. 17, 2025

(54) CONDUCTING POLYMER MICROPARTICLES AND CONDUCTING POLYMER GRANULAR HYDROGEL FOR BIOMEDICAL APPLICATIONS

(71) Applicant: Washington University, St. Louis, MO (US)

(72) Inventors: Alexandra Rutz, St. Louis, MO (US); Anna Goestenkors, St. Louis, MO (US)

(73) Assignee: Washington University, St. Louis, MO (US)

(21) Appl. No.: 18/915,345

(22) Filed: Oct. 14, 2024

Related U.S. Application Data
(60) Provisional application No. 63/590,320, filed on Oct. 13, 2023.

(51) Int. Cl. A61B 5/256; C08J 3/075; C08J 3/24

(52) U.S. Cl. CPC A61B 5/256; C08J 3/075; C08J 3/246; A61B 2560/0468; C08J 2337/00

(57) ABSTRACT

Among the various aspects of the present disclosure are the provision of conductive granular hydrogel compositions, bioelectric devices comprising the conductive granular hydrogel compositions such as wearable electrodes, conductive filaments, bioink compositions comprising living cells encapsulated in the conducting polymer composition, bioelectronic hydrogel-based devices, and methods of use thereof. The conducting 3D hydrogel is characterized by a void fraction value and high conductivity for in vitro cell applications. In addition, methods of producing the conducting 3D hydrogels and bioinks, methods of fabricating the bioelectronic hydrogel-based devices, and methods of performing bioelectronic measurements using the bioelectronic hydrogel-based devices are disclosed.

### CROSS-REFERENCE TO RELATED APPLICATIONS

This application claims priority from U.S. Provisional Application Ser. No. 63/590,320 filed on Oct. 13, 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to conducting polymer microparticles and conducting polymer granular hydrogel for biomedical applications.

### BACKGROUND OF THE INVENTION

Cell-based in vitro models aim to simulate biological environments and reactions through the growth of cells in artificial environments. This is in contrast to in vivo models which commonly utilize animal subjects. In vitro models have already been adopted for use in tissue engineering as well as drug discovery and toxicology studies due to their efficiency, cost-effectiveness, ethical advantages, and easy standardization and validation in comparison to in vivo models. In vitro models are usually evaluated with a methodology that utilizes discrete sampling where one sample is taken from a single timepoint and analyzed. These samples are also commonly sacrificial samples where once analyzed, the sample cannot be used for further study. Therefore, these methods are difficult to scale up and fail to provide real-time information. One potential solution is the use of electronic devices to convert biological signals to electronic signals which allow for efficient and high-throughput evaluation of the in vitro models in real time. Inorganic in vitro electronic devices utilize materials like gold, platinum, and silicon to interface with cell models and employ electrodes, transistors, microelectrode arrays, and other components. While these materials allow for electronic monitoring of the in vitro environments, the inorganic materials are flat and rigid. Organic in vitro electronic devices utilize materials like conducting polymers. These conducting polymers can be processed into many soft forms and are both ionically and electronically conducting. This allows them to operate at the interface of the cells and electrodes, providing a soft environment while converting biological signals to electronic signals.

While organic in vitro electronics provide a softer environment for cells than inorganic, they still fail to provide a 3D environment accompanied by precise control of cell placement and density. State-of-the-art organic in vitro electronics still commonly utilize 2D setups. The conducting polymer is deposited on top of the electrode and cells are then deposited on top of the conducting polymer. This fails to imitate the three-dimensionality of native cell environments and control the position and density of the cells interacting with the conducting polymer. Having areas of conducting polymer without any cells or with a much greater density of cells than intended makes signal interpretation more difficult. While there are some 3D porous setups for in vitro electronics where cells can infiltrate the bulk of the conducting polymer, the cells are still deposited on top of the fully formed conducting polymer structure which continues to limit control over cell placement and density. Encapsulation of cells within the conducting polymer prior to the configuration of the in vitro set-up and the use of 3D printing to deposit the cells and polymer simultaneously (i.e. 3D bioprinting) onto the electrode provide a route for creating an organic in vitro electronic that utilizes a 3D set-up and allows for precise control of cell/conducting polymer placement and cell density throughout the conducting polymer. Conducting polymer bioinks, defined herein as cells mixed and dispersed throughout a material volume, with high enough conductivity for device use are seldom reported. An important challenge is the fact that many conducting hydrogel precursors are cytotoxic and thus prohibit mixing of cells in their volume prior to gelation.

### SUMMARY OF THE INVENTION

Among the various aspects of the present disclosure are the provision of conductive granular hydrogel compositions, bioelectric devices comprising the conductive granular hydrogel compositions such as wearable electrodes and conductive filaments, and other bioelectric devices and materials comprising a plurality of living cells encapsulated within the conductive granular hydrogel composition such as 3D in vitro cell environments and bioink compositions. The conductive granular hydrogel compositions are characterized by void fraction values and high conductivities suitable for in vitro and in vivo applications. In addition, methods of producing the conductive granular hydrogel compositions and methods of fabricating the bioelectronic devices are disclosed.

In one aspect, a conductive granular hydrogel composition for biomedical applications is disclosed. The composition comprises a plurality of conductive hydrogel microparticles defining a plurality of interconnected micropores. Each hydrogel particle comprises a conductive poly(3,4-ethylene-dioxythiophene):polystyrene sulfonate (PEDOT:PSS) composite polymer, wherein the composition comprises a conductivity of about 50-140 S/m. In some aspects, the composition further comprises a void fraction value ranging from about 0.1 to about 0.95. In some aspects, the composition is further configured to encapsulate living cells. In some aspects, the composition is configured to be administered by a method selected from injection, external application, and any combination thereof. In some aspects, the conductive hydrogel microparticles further comprise a gelation agent; the gelation agent comprises an ionic liquid. In some aspects, the conductive hydrogel microparticles can be post-treated for further removal of the insulating component of the polymer, polystyrene sulfonate (PSS), resulting in an increase in conductivity. In some aspects, the conductive hydrogel microparticles further comprise a plurality of living cells, a plurality of microparticles comprising one or more therapeutics, and any combination thereof.

In another aspect, a bioelectronic device is disclosed that includes a conductive granular hydrogel composition. The composition comprises a plurality of conductive hydrogel microparticles defining a plurality of interconnected micropores, each hydrogel microparticles comprising a conductive poly(3,4-ethylene-dioxythiophene):polystyrene sulfonate (PEDOT:PSS) composite polymer, wherein the composition comprises a conductivity of about 50-140 S/m and a void fraction value ranging from about 0.1 to about 0.95. In some aspects, the bioelectric device comprises at least one wearable electrode comprising the conductive granular conducting hydrogel composition applied over an external surface of an organism. In some aspects, the bioelectric device comprises at least one conductive filament comprising the conductive granular hydrogel composition, wherein the conductive filament is formed by extruding the composition using a needle of syringe or a nozzle of a printing device. In some aspects, the bioelectric device comprises a 3D in vitro cell environment comprising a cell culture medium, and a plurality of cells embedded within the conductive granular hydrogel composition. In some aspects, the cell culture medium further comprises a stabilizing agent selected from an ionic compound comprising NaCl, an amino acid comprising tryptophan, phenylalanine or alanine, any salt thereof, and any combination thereof, wherein the stabilizing agent maintains the structure of the conductive granular hydrogel composition within the culture medium. In some aspects, the device comprises a bioink, the bioink comprising a plurality of living cells embedded in the conductive granular hydrogel composition, wherein the bioink is configured to be extruded through a nozzle of a printing device.

In another aspect, a method of fabricating a granular conducting hydrogel is disclosed that includes forming an oil phase mixture by combining a mineral oil and a surfactant; forming an aqueous phase solution comprising a poly(3,4-ethylene-dioxythiophene):polystyrene sulfonate (PEDOT:PSS) composite polymer; rapidly stirring the oil phase mixture at a temperature of about 90° C.; introducing the aqueous phase solution into the stirring oil phase mixture to form an emulsion comprising droplets of the aqueous phase solution stabilized within the oil phase mixture; gelling the aqueous phase mixture within the droplets in the stirring oil phase mixture to form conductive PEDOT:PSS hydrogel microparticles suspended in the oil phase mixture; separating the hydrogel microparticles from the oil phase mixture by rinsing with phosphate buffered saline (PBS); and packing the separated hydrogel microparticles to a predetermined void fraction value to produce the conductive granular hydrogel composition. The conductive granular hydrogel composition comprises the hydrogel microparticles packed together in a jammed state, wherein the hydrogel microparticles define a plurality of interconnected micropores. In some aspects, reducing a spacing between the separated hydrogel microparticles further comprises centrifuging the separated hydrogel microparticles, subjecting the separated hydrogel microparticles to vacuum filtration, and any combination thereof. In some aspects, the centrifuging is conducted at a centrifugal force ranging from about 2000 RCF to about 6000 RCF. In some aspects, the void fraction value ranges from about 0.1 to about 0.95 when created using centrifugation, or from about 0.1 to 0.3 when created using vacuum filtration. In some aspects, the surfactant comprises Span-80, wherein the Span-80 stabilizes the emulsion. In some aspects, the aqueous phase mixture further comprises a gelation agent, the gelation agent comprising an ionic liquid. In some aspects, the method further includes filtering the hydrogel microparticles suspended in the oil phase mixture to select a monodisperse portion of the hydrogel microparticles with diameters between 10 μm and 60 μm. In some aspects, the method further comprises comprising post-treating the hydrogel microparticles to remove at least a portion of an insulating portion of the composite polymer comprising PSS, wherein the post-treated conducting hydrogel composition comprises a conductivity of about 50-140 S/m.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a schematic illustration showing a method of producing PEDOT:PSS microparticles using water-in-oil emulsion. A PEDOT:PSS precursor solution is added to an oil phase consisting of mineral oil and 0.05% (weight %) Span-80 while stirring at 750 RPM.

FIG. 2A is a set of images summarizing the effect of the oil phase temperature used in the process illustrated in FIG. 1 the impacts the size of the PEDOT:PSS phase droplets following deposition into the oil phase. Over 60 minutes, the PEDOT:PSS phase droplets at 90° C. decreased in size.

FIG. 2B is a set of graphs summarizing mean droplet diameter measurements over time at the three oil phase temperatures shown in FIG. 2A that confirmed that oil phase temperatures of 25° C. and 60° C. showed no significant difference in droplet diameter at later timepoints in comparison to the droplet diameter at 0 minutes. Droplets formed using an oil phase temperature of 90° C. showed significant differences between droplet diameter at all later time points in comparison to 0 minutes. 10 droplets were measured per time point within each condition. One-way analysis of variance (ANOVA) and Tukey's multiple comparison test. ****P≤0.0001 ***P≤0.001 **P≤0.01 *P≤0.05 non-significant (NS) P>0.05.

FIG. 3A is a set of images showing that emulsions conducted with oil phase temperatures of 60° C. and 90° C. resulted in aqueous stable PEDOT:PSS microparticle formation after de-emulsification. Emulsions conducted with an oil phase temperature of 25° C. did not result in microparticle formation as only mineral oil droplets were observed.

FIG. 3B is a graph of mean particle diameter of particles formed as shown in FIG. 1 at different oil phase temperatures. An oil phase temperature of 60° C. resulted in the formation of microparticles with an average diameter of 50.13 micrometers, and an oil phase temperature of 90° C. produced microparticles with an average diameter of 74.50 micrometers.

FIG. 3C is a graph of polydispersity index (PDI) of microparticles formed as shown in FIG. 1 at different oil phase temperatures. An oil phase temperature of 90° C. produced microparticles with a PDI of 0.0725, indicating a monodisperse microparticle population. An oil phase temperature of 60° C. produced microparticles with a PDI value of 0.2178, indicating a more polydisperse population.

FIG. 4A is an image of aqueous stable PEDOT:PSS microparticles that were generated from PEDOT:PSS.

FIG. 4B is an image of aqueous stable PEDOT:PSS microparticles that were generated from PEDOT:PSS in combination with a gelation agent dodecylbenzene sulfonic acid (DBSA)

FIG. 4C is an image of aqueous stable PEDOT:PSS microparticles that were generated from PEDOT:PSS in combination with an ionic liquid (IL) gelation agent, 4-(3-butyl-1-imidazolio)-1-butanesulfonic acid triflate.

FIG. 5A is a pair of images illustrating size selection of a microparticle population using filtration of defined pore sizes to adjust mean diameter of the microparticles. Prior to filtration (top), the microparticle population has less uniformity with an average diameter of 59.44 micrometers and a range of diameters between 10 and 200 micrometers resulting in a polydispersity index of 0.3141. After filtration (bottom) using filters to isolate microparticles between 10 and 60 micrometers, the population possesses greater uniformity and an average diameter of 37.56 micrometers and a range of diameters between 10 and 60 micrometers resulting in a polydispersity index of 0.1027.

FIG. 5B is a graph summarizing the diameters and PDIs of the microparticle populations before and after filtration as described in FIG. 5A.

FIG. 6A is an image showing the microparticles suspended in an aqueous phase (water or phosphate buffered saline) following fabrication as illustrated in FIG. 1 to form a microparticle suspension. Centrifugation or vacuum filtration is used to remove a large portion of the aqueous phase and pack the microparticles close together to form a granular hydrogel.

FIG. 6B is an image of a granular hydrogel formed from the suspended microparticles of FIG. 6A after centrifugation or vacuum filtration.

FIG. 6C is an image of the granular hydrogel of FIG. 6B. After centrifugation or vacuum filtration, the “jammed state” of the microparticles have non-specific interactions that maintain the densely packed state of the microparticles, resulting in a “paste-like” material that can be handled more like a bulk hydrogel.

FIG. 7 is an image showing a granular hydrogel packed densely using vacuum filtration. The granular hydrogel possesses an average pore diameter of 17.96 micrometers and a range of pore diameters from 5.13 to 38.75 micrometers. The void space of the granular hydrogel is visualized in the image by introducing fluorescein isothiocyanate-dextran into the void space to fluoresce the pores (green) defined between adjacent microparticles (black). Pore diameter is measured by quantifying the longest distance in a pore between adjacent microparticles.

FIG. 8 is a set of images showing that when densely packed, the granular hydrogel can be extruded through a 3D printing nozzle with an inner diameter of 580 micrometers (left) and used to form various three-dimensional configurations such as a filament (center) or a sphere (right).

FIG. 9A is a graph of viscosity as a function of shear rate measured from a granular hydrogel material sample.

FIG. 9B is a graph of storage modulus and loss modulus as a function of oscillation strain.

FIG. 9C is graph of storage modulus and loss modulus.

FIG. 10A is a pair of images of extruded strands of granular hydrogel formed using lower (2000 RCF, left) and higher (5400 RCF, right) centrifugal forces

FIG. 10B is a bar graph comparing the mean free path of the granular hydrogel materials of FIG. 10A.

FIG. 11 is a pair of images showing a side view of collagen hydrogel overlay applied over the top of the granular hydrogel in a Petri dish (left) and a top view (right) of the hydrogel after water was applied over the top of the collagen overlay.

FIG. 12A contains a set of images of petri dishes containing filaments of the granular hydrogel extruded into the solution via a 20-gauge nozzle into various aqueous solution compositions.

FIG. 12B contains a set of images of petri dishes containing filaments of the granular hydrogel extruded via a 20-gauge nozzle into various aqueous solution compositions that include phosphate buffered saline with various amino acids added at concentrations of 0.5% and 1%: tryptophan (left column), phenylalanine (center column), and alanine (right column).

FIG. 13A is a set of images showing normal human dermal fibroblasts encapsulated within the microparticles of a granular hydrogel material and cultured for 14 days. Green cells indicate living cells and red cells indicate dead cells. Maximum intensity projections of samples obtained through confocal imaging show live cells are maintained in a greater number than dead cells.

FIG. 13B is a graph summarizing an analysis of the images of FIG. 13A, indicating a cell viability of greater than 80% over the 14 days of culturing.

FIG. 14A is a schematic showing that the granular hydrogel bioink can be used to create 3D in vitro environments. This can be done by the encapsulation of cells within the material

FIG. 14B is a schematic showing how 3D printing of the now bioink onto an electrode is performed.

FIG. 15 is a bar graph comparing the conductivity of granular conducting hydrogels containing PEDOT:PSS microparticles, PEDOT:PSS/IL microparticles, and post-treated PEDOT:PSS/IL microparticles.

### DETAILED DESCRIPTION OF THE INVENTION

The present disclosure is based, at least in part, on the discovery that a granular conducting hydrogel with functional void fraction and a conductivity of about 50-140 S/m may be used to encapsulate cells in a bioink composition that may be used to produce 3D printed bioelectronic devices.

As shown herein, conducting polymer granular hydrogel bioinks for 3D printed in vitro bioelectronic devices, as well as their use in high throughput electrophysiology, are described.

One aspect of the present disclosure provides for the creation of a granular conducting polymer bioink composition. In another aspect, the conducting granular polymer hydrogel formed using the disclosed bioink composition can provide a soft, cell-encapsulating environment under cytocompatible conditions. Additionally, the granular conducting polymer hydrogels possess a hydrated nature that provides for the diffusion necessary for cell viability should this form be used for a bioink and include encapsulated cells.

In one aspect, the devices incorporating 3D in vitro environments may be formed by 3D printing cells encapsulated in the conducting granular hydrogel material disclosed herein. Granular hydrogels have previously been used in 3D bioprinting. Soft micron-scale hydrogels, also known as microgels, have been fabricated from non-conducting polymers. When these soft microgels are put into a densely packed state they are known as a granular hydrogel. Their interactions allow for the material to flow when put under shear force (force generated when 3D printing) and recover when shear force is removed. These attributes make them ideal for 3D printing various shapes and configurations and maintaining that shape and configuration once completed, respectively. The granular hydrogel is inherently microporous, containing an interconnected microporous structure, which allows for cells to be added to the material. That porous space is referred to as the void fraction of the granular hydrogel, defined herein as a quantitative measure of the amount of porous volume present in the hydrogel defined as the ratio of the porous space volume to the entire granular hydrogel volume. The granular hydrogel and cells can then be 3D printed together. The cells are protected within the pores of the material and by the material's ability to flow under shear force during the printing process.

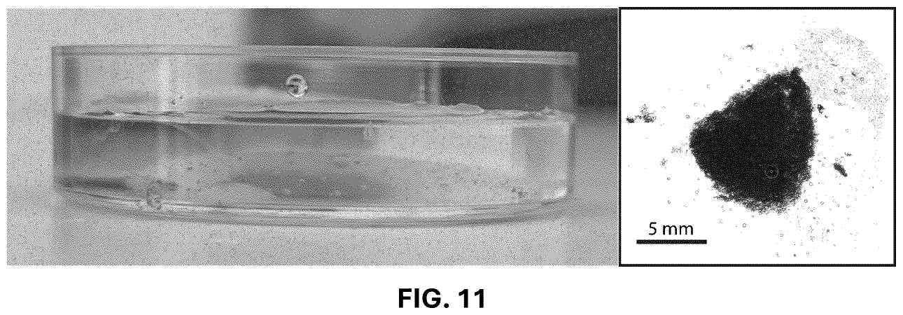

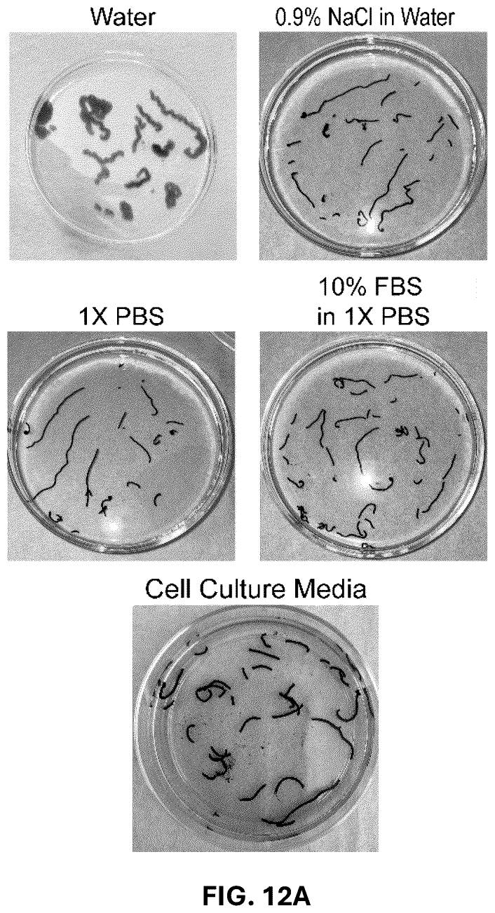

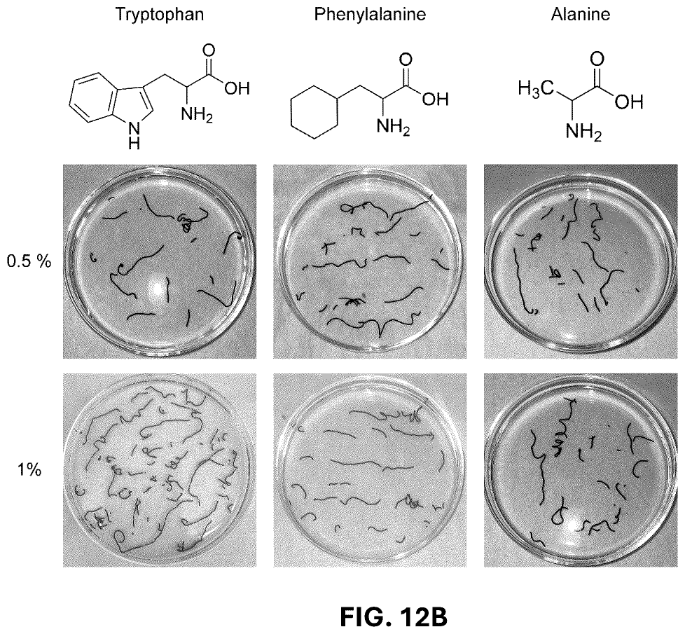

It was hypothesized conducting polymer could be used to fabricate conducting microgels which could then be put into a densely packed state to become a conducting granular hydrogel. The ability to encapsulate cells within the granular hydrogel and then 3D print both components can make the material a conducting granular hydrogel bioink. When the conducting granular hydrogel bioink comes into contact with a conducting path, usually one of metal substrate or wire, the configuration then becomes an electrode device. Through this connection, the electrode can be used for recording, stimulating, or sensing purposes; generally proposed for measuring or influencing biological phenomena. 3D bioprinting can be used to place the conducting granular hydrogel and encapsulated cells onto the conducting path with great precision. The granular hydrogel and cells need to be stabilized on the conducting path. In one aspect, the granular hydrogel may be stabilized through the application of a collagen hydrogel that covers the top surface of the granular hydrogel without penetrating the structure underneath, as illustrated in FIG. 11. In other aspects, the granular hydrogel may be stabilized by including compounds with aromatic rings and/or charged species such amino acids like phenylalanine or tryptophan or salts thereof in the surrounding cell culture solution as illustrated in FIG. 12B. Without being limited to any particular theory, this inclusion of aromatic amino acids and salts thereof is thought to create non-covalent reversable cross-linking in the hydrogel. In some aspects, the aromatic amino acids and salts thereof can be added in an amount between 0.5-5% concentration of the overall composition of the hydrogel.

Important features of this device include the use of a conducting polymer, cell encapsulation within the material, subsequent creation of a 3D in vitro environment for biological and device advantages, and the conducting granular hydrogel's printability.

Conducting polymers can be processed into many soft forms that are both ionically and electronically conducting. This allows the polymers to operate at the interface of the cells and electrodes, providing a softer environment than existing interface materials (gold, platinum, and silicon). This softer environment better matches the softness of the native tissue environment of cells. At the interface of the cells and electrodes, the conducting polymers are also able to aid in converting biological signals to electronic signals due to their dual conductivity. The conducting polymer can be used with a carrier polymer, but it is not required in order to facilitate the formation of the material or 3D bioprinting. Exclusion of the carrier polymer maintains a high density of the conducting polymer network surrounding the cells. The encapsulation of the cells within the granular hydrogel allows for the creation of a 3D in vitro environment in which cells are surrounded by the conducting polymer and precisely deposited upon the conducting path. The distance between cell signals and the conducting material is reduced in this encapsulating, three-dimensional configuration, therefore potentially resulting in less noise and causing an overall increase in the signal-to-noise ratio. It is anticipated this electronic conductivity will enable one to perform electrical recording, stimulation, and sensing by connecting the conducting path to a controller, electrophysiology equipment, and other systems. Encapsulation of cells in the material prior to 3D bioprinting protects cells from stress experienced during the printing process and allows for the creation of a 3D in vitro environment. Cells undergo shear stress during the printing process. That shear stress will be greater if the cells are encapsulated in a material that resists flow. The conducting granular hydrogel bioink is shear-thinning and self-healing meaning that when shear force is applied, the material flows and when shear force is removed the material recovers its structure. The encapsulated cells sit within the pores between the individual microgels and are protected from shear stress by the material's ability to flow under shear force. The 3D environment created by cell encapsulation and subsequent 3D printing also much more closely mimics the native cellular environment. Because these 3D cultures better represent native environments, the data procured from these models is thought to be more predictive of cellular response within the body.

The use of cells encapsulated within a shear-thinning and self-healing material also allows for the deposition of conducting polymer and cells through 3D bioprinting. When cells are deposited on top of conducting polymer by hand, it does not allow for precise placement of different cell types on specific portions of the electrode. 3D bioprinting allows for precise placement of the granular hydrogel with one incorporated cell type on one part of the electrode while granular hydrogel containing a different cell type could be precisely deposited at a different location.

In some aspects, the conducting polymer formulation may include commercially available Heraeus Clevios PH1000 PEDOT:PSS (poly(3,4-ethylenedioxythiophene) polystyrene sulfonate). It was hypothesized that one could use water-in-oil emulsion to create micro-sized droplets of PEDOT:PSS that afterward could cause them to solidify into microgels before de-emulsification. Since PEDOT:PSS can be synthesized as an aqueous colloidal dispersion, its aqueous nature would cause the immiscibility of the polymer with oil. Therefore, the idea was to disperse the polymer solution into oil such that micro-sized droplets were generated and were stable long enough to enable gelation to occur prior to de-emulsification.

In various aspects, a process to create highly spherical microgels is disclosed that includes forming an aqueous phase comprising a conducting polymer comprising poly(3,4-ethylene-dioxythiophene):polystyrene sulfonate (PEDOT:PSS) composite polymer. This aqueous phase is deposited into an oil phase being stirred at a high speed to create a water-in-oil emulsion comprising micron-scale polymer/gelation agent spherical droplets encapsulated within and stabilized by the oil phase. The emulsion is exposed to an increased temperature condition that enables and accelerates gelation. Without being limited to any particular theory, the increased temperature level influences the gelation, structure, and functional properties of the resulting conducting granular hydrogel. In various aspects, the particular temperature employed plays an important role in the process. In some aspects, the formation of the spherical droplets within the oil phase is conducted at a temperature ranging from about 80° C. to about 110° C. In other aspects, the formation of the spherical droplets within the oil phase is conducted at a temperature ranging from about 90° C. to about 100° C. In one exemplary aspect, the formation of the spherical droplets within the oil phase is conducted at a temperature of about 90° C.

In various aspects, the aqueous solution containing PEDOT:PSS may further comprise additional gelation agents including, but not limited to, an ionic liquid, dodecylbenzene sulfonic acid (DBSA), and any other suitable gelation agent without limitation. DBSA is another gelation agent triggered by increased temperatures commonly used to make bulk PEDOT:PSS hydrogels. In some embodiments, the ionic liquid may further influence the conductivity of the resulting granular hydrogel. In some embodiments, the post-treatment of microgels for removal of insulating polystyrene sulfonate will result in an increase in conductivity up to 140 S/m.

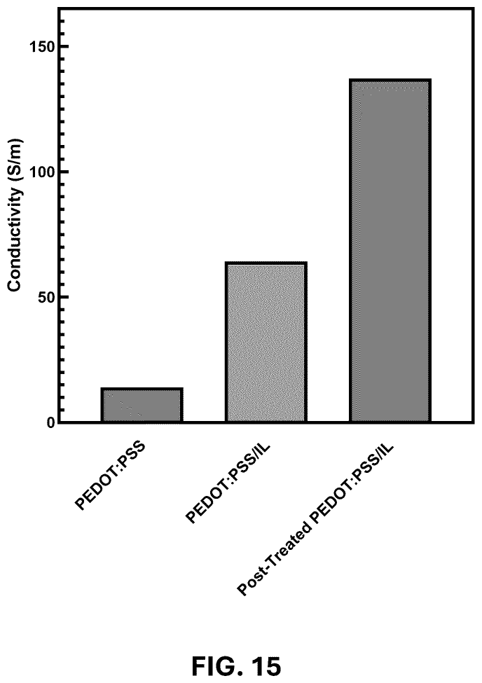

By way of non-limiting example, several conductive granular hydrogels were formed with PEDOT:PSS microparticles, PEDOT:PSS/IL microparticles, and PEDOT:PSS/IL microparticles post-treated to remove at least a portion of the non-conductive PSS portion of the composite conductive polymer. The conductivities of each conductive granular hydrogel material were measured and compared. As summarized in FIG. 15, the granular hydrogel materials had conductivities of 14.08 S/m, 64.25 S/m, and 137.23 S/m, respectively.

When the micron-scale polymer deposits are gelled within the mineral oil, the mineral oil is removed through repeated washes with phosphate-buffered saline solution. The resulting conducting polymer micron-sized spheres can then be closely packed through the removal of the phosphate-buffered saline solution either through centrifugation or vacuum filtration, producing a conducting granular hydrogel material. Various types of cells can then be added to this packed configuration for 3D bioprinting onto pre-made conducting paths.

It is also disclosed that centrifugal force can be used to tune the granular hydrogel void fraction which in turn impacts conductivity and printability. Increased centrifugal force more tightly packs the microgels. This results in less porosity or a decreased void fraction. A decreased void fraction increases conductivity and increases printability as demonstrated by increased maximum filament length and the filament diameter better matching the chosen nozzle diameter. Rheological investigations confirmed the material flowed under increased shear force (shear-thinning) and recovered when shear force was removed (self-healing). Normal human dermal fibroblasts have been encapsulated in the PEDOT:PSS granular material and high cell viability was maintained for fourteen days. The granular material was successfully 3D printed into various three-dimensional configurations and those structures have been maintained with a collagen hydrogel overlay for at least ninety days. The conductivity of the granular materials at the lowest void fraction achieved utilizing centrifugation and vacuum filtration were also evaluated and found to be 50-140 S/m.

In some aspects, a relatively high void fraction lends the material for easy injectability, with larger particles, and larger pores. In another aspect, the pore size can influence cell movement and immune cell recruitment. In yet another aspect the pore size can be tuned to fit the intended applicable function of the material. In accordance with another aspect a relatively low void fraction, which is characterized by relatively small pores and small particles. In another aspect, this relatively low void fraction creates a higher conductivity compared to a high void fraction counterpart. In another aspect, these smaller particles create higher resolution in 2D/3D printing applications.

In some aspects, the hydrogel microparticles can be densely packed using centrifugation. Without being limited to any particular theory, the level of centrifugal force used influences void fraction, conductivity, and extrudability of the resulting granular hydrogels. In other aspects, the microparticles can be densely packed with vacuum filtration. Centrifugation can be used to create granular hydrogels with a void fraction range of 0.1 to 0.95. At the highest centrifugal force and lowest void fraction, the conductivity of granular hydrogels made with centrifugation is 50-140 Siemens per meter. Granular hydrogels created with vacuum filtration have a void fraction range of 0.1 to 0.3 and a conductivity of about 50-140 Siemens per meter.

A granular hydrogel is governed by non-specific physical interactions, mainly gravitational and frictional forces. These interactions are so weak that they can be easily disrupted by weak mechanical forces such as fluid flow. Therefore, to utilize the granular hydrogel in aqueous environments, such as cell culture where media is required to keep cells alive, stronger and/or more permanent forces are required. In some aspects, extrusion of the granular hydrogel into a solution containing a salt including to but not limited to, sodium chloride solution or phosphate buffered saline, and an amino acid including, but not limited to, alanine, phenylalanine, and tryptophan, stabilizes the extruded strands. Without these additional stabilizing forces, the granular hydrogel would resuspend into a microparticle suspension and lose the 3D printed structure. Therefore, the methods described herein facilitate 3D printing of the granular hydrogel and encapsulated cells onto electrodes submerged in these solutions to create the 3D cell laden electrodes and maintain material stability throughout the entirety of cell culture.

By way of non-limiting example, PEDOT:PSS microgels were made with 10 mg/ml of ionic liquid as described herein, densely packed through vacuum filtration, and extruded into solutions via a 20 G tapered nozzle (580 micrometer inner diameter). The granular hydrogel was syringe extruded through a nozzle into physiological solutions into various aqueous environments, as illustrated in the images of FIG. 12A. Stabilization of the extruded hydrogel filaments was achieved in a saline solution (0.9% NaCl) with and without phosphate buffer (PBS), phosphate buffered saline (PBS) with fetal bovine serum (FBS), and cell culture media (Lonza Fibroblast Growth Media). Images shown in FIG. 12A were obtained within minutes after extrusion of the granular hydrogel into the various solutions. Extruded strands were then monitored for a minimum of 24 hours and confirmed to stabilize the strands over this time period.

By way of another non-limiting example, PEDOT:PSS microgels were made with 10 mg/mL of ionic liquid as described herein, densely packed through vacuum filtration, and extruded into solutions via a 20 G tapered nozzle (580 micrometer inner diameter). The granular hydrogel was syringe extruded through a nozzle into phosphate buffered saline containing one of three different amino acids at concentrations of 0.5% and 1%. Images shown in FIG. 12B were obtained within minutes after extrusion of the granular hydrogel into the various solutions. Extruded strands were then monitored for a minimum of 24 hours and all conditions shown were confirmed to stabilize strands over this time period.

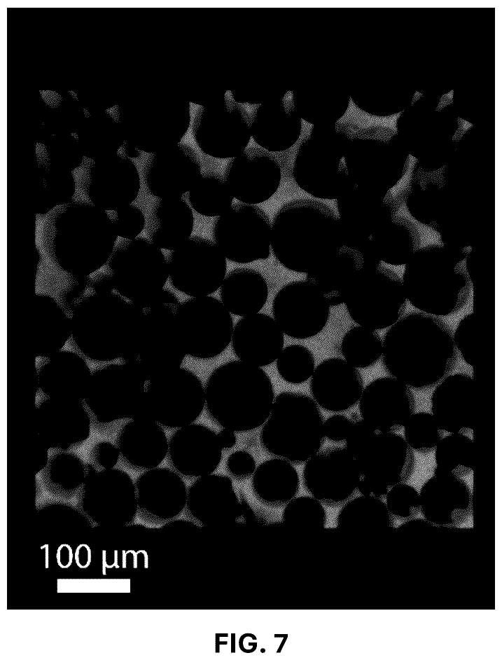

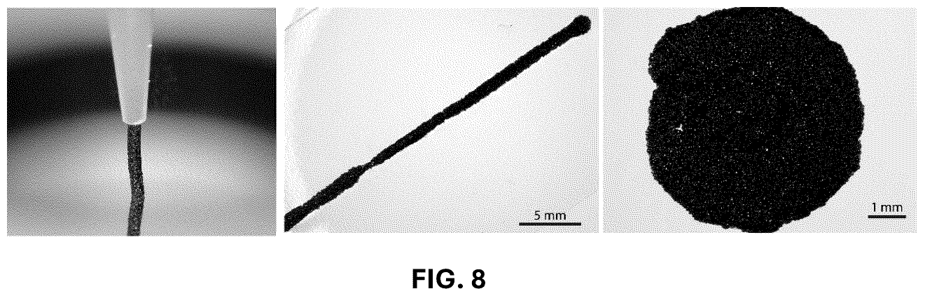

In various aspects, the granular hydrogel comprises an interconnected microporous structure, as illustrated in FIG. 7. In some aspects, the interconnected microporous structure includes encapsulated cells and the interconnected micropores provide for the diffusion of nutrients and waste to and from the granular hydrogel which is necessary for encapsulated cells. In one exemplary aspect, the granular hydrogel comprises interconnected micropores throughout the structure with average pore diameters of 17.96 micrometers and a range of 5.13 micrometers to 83.75 micrometers.

In other aspects, the hydrogel material is provided as an injectable formulation. Non-conducting granular hydrogels are commonly used as injectables either with or without encapsulated cells to encourage cell and tissue growth in wounds. Without being limited to any particular theory, it is thought that the injection of the granular hydrogel into an organism facilitates cell recruitment and vascularization. In addition, the PEDOT:PSS microparticles can be mixed with microparticles containing therapeutics for biological function in addition to the conductivity of the PEDOT:PSS granular hydrogel. For example, microparticles composed of polymers with cell-adhesive motifs can be mixed with the PEDOT:PSS microparticles and then jammed to create a heterogeneous granular hydrogel. When injected into wounds, the adhesive motifs can encourage cell migration and the PEDOT:PSS granular hydrogel can be used to monitor and/or stimulate cell activity within the wound. In some aspects, the material can be formulated as an injectable. In some aspects, the injectable form of the granular hydrogel is suitable for delivery through the needle of a syringe. By way of non-limiting example, the needle of a syringe containing the granular hydrogel is inserted into a tissue and used to form a wire by injecting the granular hydrogel composition as the needle is pulled out.

In one aspect, the hydrogel material can be used as a wearable electrode. The granular hydrogel can conform to topographically diverse structures due to the non-specific physical forces that govern the material. Therefore, it can be placed on tissues and used to monitor electronic signals. In other aspects, the hydrogel material can be used for both in vitro and in vivo applications.

In the present disclosure, it is shown that elevated temperature is required for microparticle formation within 60 minutes of PEDOT:PSS addition to the oil phase. Microparticles did not form within 1 hour when PEDOT:PSS was added to 25° C. mineral oil. In addition, microparticles formed at 90° C. were monodisperse with a polydispersity value of 0.0725 while microparticles formed at 60° C. were polydisperse with a polydispersity value of 0.2178. In one exemplary aspect, the fabrication temperature is about 90° C.

In addition, the conductivity of microparticles made with PEDOT:PSS colloidal dispersion in the absence of a gelation agent is inconsistent and relatively low in comparison to microparticles fabricated with PEDOT:PSS with 10 mg/ml of ionic liquid. The conductivity of the granular hydrogel is higher and more consistent between samples with an average conductivity of 50.96 Siemens per meter and a standard deviation of 1.47 Siemens per meter. Post-treatment of PEDOT:PSS microparticles fabricated with 10 mg/mL for removal of insulating polystyrene sulfonate (PSS) causes a further increase in conductivity with an average conductivity of 137.23 S/m.

Once the material and encapsulated cells are printed onto the conducting path the device is proposed for in vitro use. A number of applications have been proposed and explained in greater detail in the Examples. One example includes the use of the 3D in vitro setup to culture and record data from 3D cell cultures including neural organoids. Neural systems are possible models for predicting personalized responses to stressors, drug screening, and studying neuromodulation. Neural organoids are typically spherical with large diameters which greatly limits the cell-electrode surface area in 2D set-ups. Neural organoids can also flatten in 2D set-ups and lose their characteristic properties of organoid culture. The organoids can be encapsulated in the granular material and then 3D printed onto a conducting path. The granular hydrogel would surround the entirety of the organoid and greatly increase the cell-electrode surface area. The organoid and material can also be precisely deposited directly on the conducting path. This is anticipated to generate better signal properties during recording and stimulation. Additionally, the ability to 3D print the granular hydrogel bioink allows for multi-material printing on the same electrode. Various cell types can be incorporated into separate samples of the granular hydrogel and printed sequentially onto different portions of the same electrode. This can be used to monitor various cellular responses to stimulation from the same electrode or to build a heterogeneous cellular environment more closely matching native cellular environments.

The present disclosure presents a new type of conducting granular hydrogel bioink for use in the creation of a bioelectronic device-one that is potentially better suited for in vitro cellular and tissue interfacing. The granular hydrogel is the first to be comprised of highly spherical, monodisperse pure conducting polymer microgels that can be densely packed to form the granular hydrogel. The conducting polymer microgels are softer than current state-of-the-art materials used (gold, platinum, and silicon). The microgels better match the softness of the native tissue environment of cells. The microgels are also ionically and electronically conducting allowing for them to aid in converting biological signals to electronic signals.

Cells can be encapsulated in the granular hydrogel and both components can be 3D printed making it a conducting granular hydrogel bioink. When printed on top of a conducting path, usually a metal substrate, the configuration becomes an electrode device. The conducting polymer can be but is not required to be blended into a carrier polymer in order to facilitate the formation of the material or 3D bioprinting. Exclusion of a carrier polymer maintains a high density of the conducting polymer network surrounding the cells.

Additionally, the encapsulation of cells in the material prior to 3D bioprinting allows for the creation of a 3D in vitro environment in which cells are surrounded by the conducting polymer and precisely deposited upon the conducting path. This may be ideal for organoid or tissue culture where the 3D printed electrode may serve to measure some biological phenomena or stimulate the tissue for enhanced culture (e.g. promote or accelerate differentiation of stem cells). The encapsulation of the cells in the material prior to 3D bioprinting also allows for precise placement of the material and the cells on the metal electrode in comparison to current methods that involve hand placement of materials and cells. The distance between cell signals and the conducting material is also reduced in this encapsulating, three-dimensional configuration, therefore potentially resulting in less noise and causing an overall increase in the signal-to-noise ratio. It is anticipated this electronic conductivity enables one to perform electrical recording, stimulation, or sensing by connecting the conducting path to a controller, electrophysiology equipment, and other systems.

Encapsulation of cells in the material prior to 3D bioprinting protects cells from stress experienced during the printing process and allows for the creation of a 3D in vitro environment. Cells undergo shear stress during the printing process. That shear stress is greater if the cells are encapsulated in a material that resists flow. The conducting granular hydrogel bioink is strain yielding, shear-thinning, and self-healing meaning that when shear force is applied, the material flows and when shear force is removed the material recovers its structure. The encapsulated cells sit within the pores between the individual microgels and are protected from shear stress by the material's ability to flow under shear force. The 3D environment created by cell encapsulation and subsequent 3D printing also much more closely mimics the native cellular environment. Because these 3D cultures better represent native environments, the data procured from these models is thought to be more predictive of cellular response within the body.

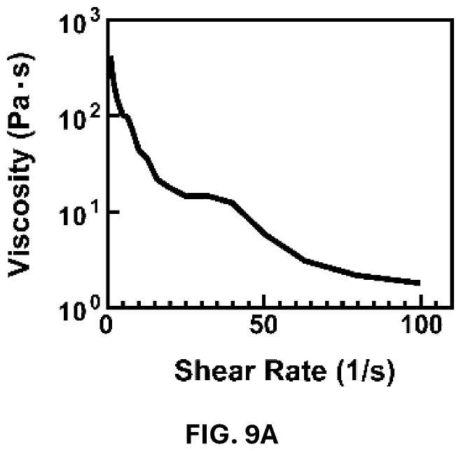

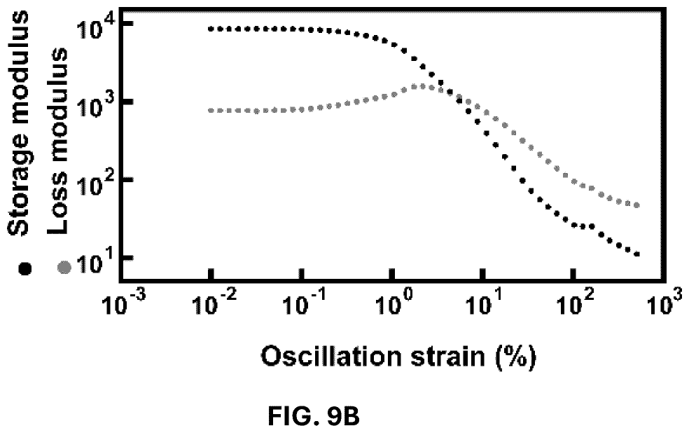

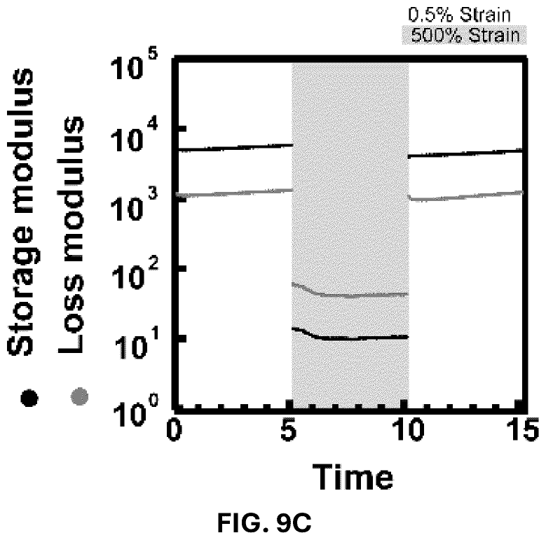

Encapsulation of cells in the material prior to injection into an organism to encourage cell recruitment, cell proliferation, and tissue regeneration in wounds protects cells from stress experienced during the injection process. Encapsulation minimizes shear stress experienced by cells during the injection process as the conducting granular hydrogel is strain-yielding, shear-thinning, and self-healing. When the shear force is applied, the material flows, and when the shear force is removed, the material recovers its structure (FIGS. 9A, 9B, and 9C). The encapsulated cells remain within the pores and are protected from shear stress.

The use of a conducting granular hydrogel bioink for in vitro device creation allows for the deposition of conducting polymer and cells through 3D bioprinting. When cells are deposited on top of conducting polymer by hand, it does not allow for precise placement of different cell types on specific portions of the electrode. 3D bioprinting allows for precise placement of the granular conducting hydrogel with one cell type incorporated on one region of the electrode while the granular conducting hydrogel incorporating a different cell type could be precisely deposited on a different region of the electrode.

### Kits

Also provided are kits. Such kits can include the conductive hydrogel disclosed herein, reagents used to form the conductive hydrogel disclosed herein, and, in certain embodiments, instructions for producing or using the conductive hydrogel as described herein. Such kits can facilitate the performance of the methods described herein. When supplied as a kit, the different components of the composition can be packaged in separate containers and admixed immediately before use. Components include, but are not limited to electrodes, a 3D conducting hydrogel and/or at least a portion of the reagents used to produce the 3D conducting hydrogel, and/or at least one type of cell. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit. The kits can be provided as starting materials, an aqueous suspension, or in lyophilized form.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Conducting Polymer Granular Hydrogel Bioinks for 3D Printed In Vitro Bioelectronic Devices

Conjugated polymers have recently been used in their hydrogel form for the creation of soft bioelectronics with improved cell interfacing and the potential for enhanced measurement and stimulation of cellular activities. Cell-encapsulating hydrogels offer a truly 3D culture environment and when designed appropriately can be used as bioinks for 3D printing which grants spatially controlled deposition of cells and materials simultaneously

To develop a method of producing the granular hydrogel form as disclosed herein, the following experiments were conducted. A bioink based on the conducting polymer poly(3,4-ethylene-dioxythiophene):polystyrene sulfonate (PEDOT:PSS) was developed by creating a granular hydrogel form. Emulsion methodology was developed to fabricate PEDOT:PSS hydrogel microparticles (microgels) with high monodispersity. Filtration techniques were used to size select microgels with diameters between 10 and 60 μm. When centrifuged to remove significant water, the conducting polymer microgels achieved dense packing characteristics of a granular material state. Increasing centrifugal force decreased the void fraction of the material which increased conductivity. Rheological investigations confirmed shear-thinning and self-healing properties, both ideal for 3D bioprinting for extrusion and the ability to keep shape, respectively. Granular PEDOT:PSS hydrogels were 3D printable via pneumatic extrusion and formed various 3D configurations. Evaluation of the material's printability in the granular state also revealed that increased centrifugal force facilitated better printability as evidenced by increased maximum filament length and filament diameter better matching the chosen nozzle diameter. Structural stability of the material within an aqueous environment for up to three months was achieved using a collagen hydrogel overlay. Human dermal fibroblasts encapsulated and cultured within the granular hydrogel showed high cell viability over fourteen days demonstrating cytocompatibility. This developed conducting granular hydrogel bioink exhibits balanced properties of printability, conductivity, and cellular responses for the additive manufacturing of future in vitro bioelectronics.

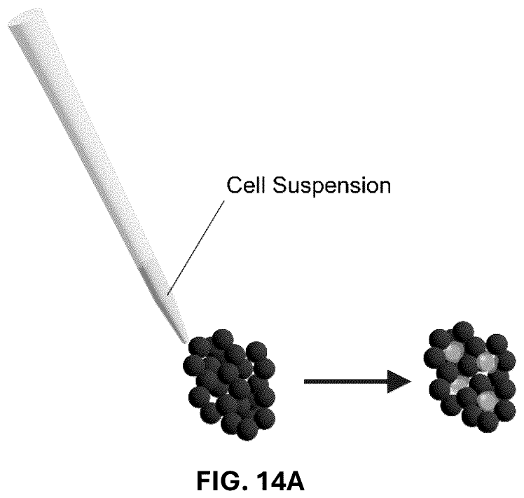

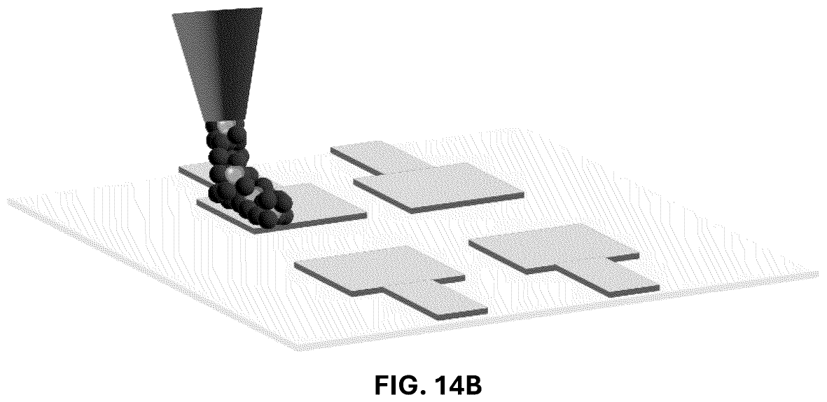

Creating a 3D cellular environment, as seen in FIG. 14, where the cell is surrounded by the electrode, enhances the surface area and results in less travel for the signals leading to the hypothesized improvements in signal amplitude and signal-to-noise ratio. In general, a hydrogel microparticle suspension includes micron-sized spheres of crosslinked polymer suspended in water. The removal of the aqueous phase puts the spheres into a “jammed state” where they are considered a granular hydrogel. In this jammed state, the spheres are shear-thinning and therefore, can be extruded as strand-like structures or filaments.

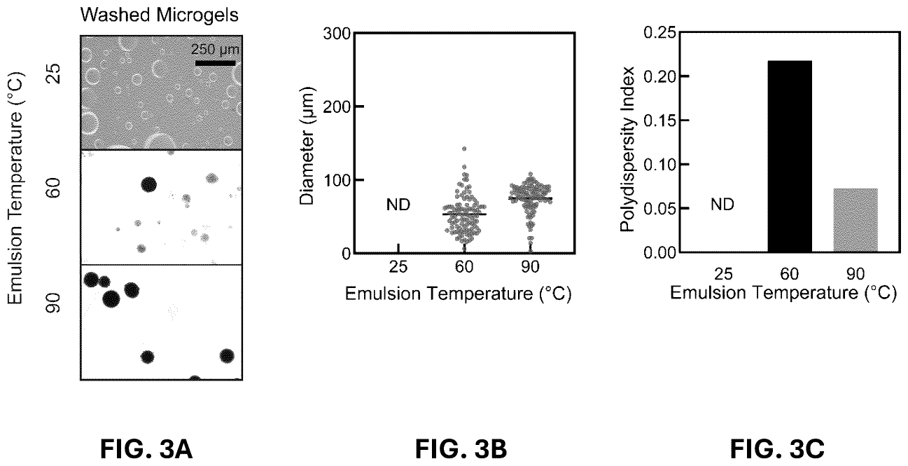

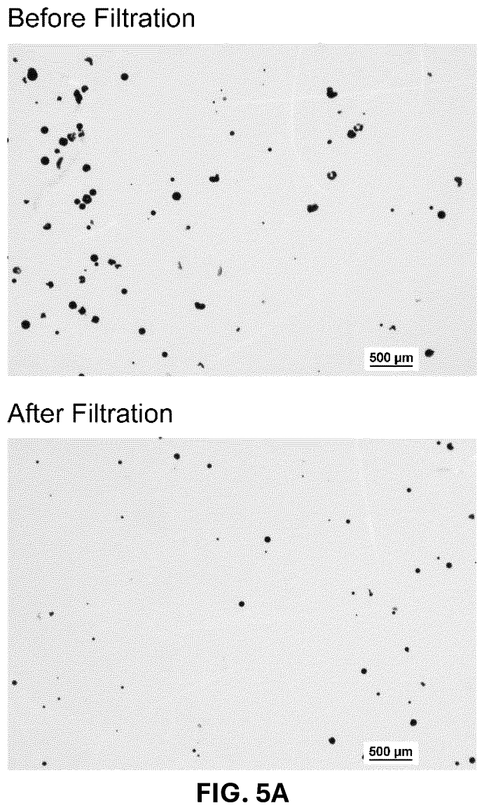

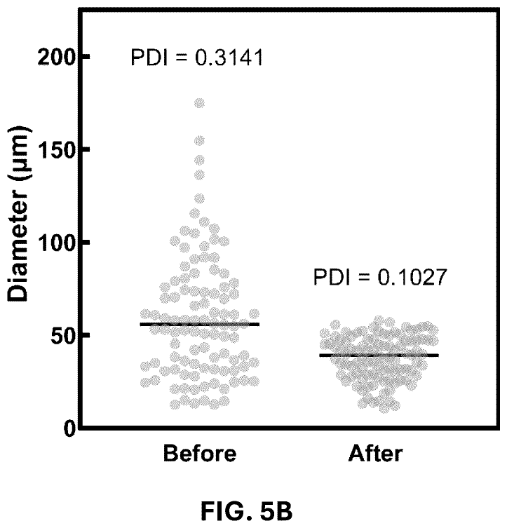

In one experiment, 4-(3-Butyl-1-imidazolio)-1-butanesolufonic acid, an ionic liquid (IL), is used as a gelation agent for PEDOT:PSS. The IL forms electrostatic interactions with PSS, exposing the PEDOT chains and promoting their interchain interactions. In this emulsion, where two immiscible liquids are mixed to produce microdroplets, a PEDOT:PSS/IL mixture (aqueous phase) is distributed using a 34G syringe into mineral oil and 0.05% Span-80 (wt %) mixture (oil phase). The emulsion was heated to 90° C. which was shown to gel PEDOT:PSS/IL thin films and stirred at 600 RPM during the addition and gelation of PEDOT:PSS/IL. This gelation is assumed to occur in one hour. After gelation, the hydrogel spheres are separated from the oil phase and washed with phosphate-buffered saline solution (PBS). Using this protocol, uniformly shaped hydrogel microspheres have been produced (FIG. 3). To produce a uniformly sized population, a 60 μm cell strainer was used to filter fragments and spheres above 60 μm, leading to a max diameter of 59.6 μm with an average diameter of 38.3 μm (N=300) (FIG. 5). The consistent size achieved is important not only for reproducibility but for creating a consistent path of spheres in a filament to allow for electronic signals to travel throughout the entire material. Removal of the aqueous phase allowed for extrusion of the material as filaments (FIG. 8).

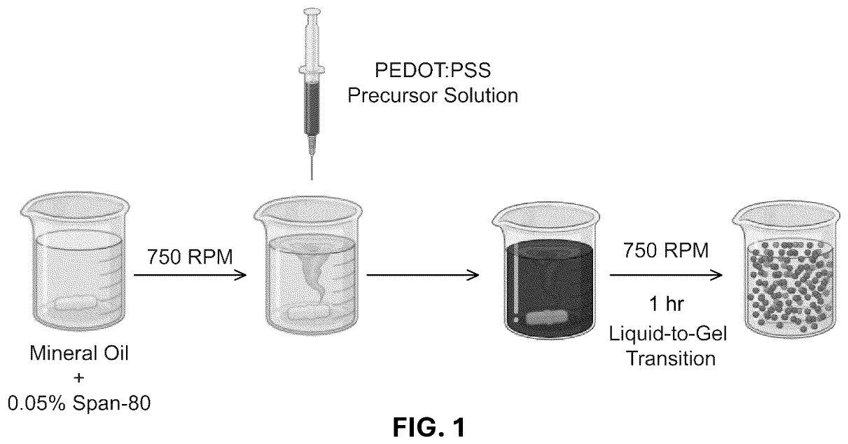

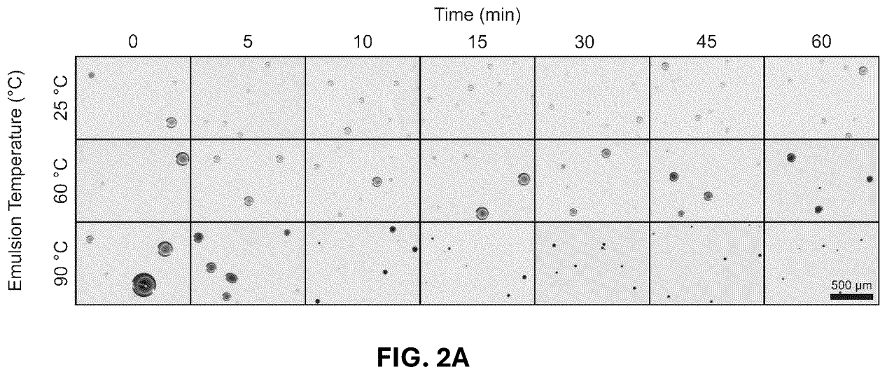

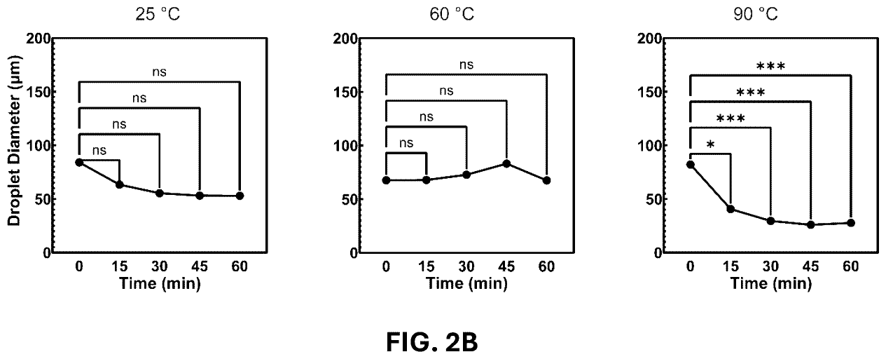

In another set of experiments, a water-in-oil emulsion is used to create spherical droplets of PEDOT:PSS and ionic liquid within mineral oil/Span-80. As seen in FIG. 1, Span-80 is added to the mineral oil to aid in stabilizing the emulsion. This oil phase is heated to 90° C. and stirred at 750 RPM. The PEDOT:PSS precursor solution is added as the aqueous phase to the oil phase. The rapid stirring breaks the aqueous phase up into micron-scale precursor droplets stabilized within the oil phase. The increased temperature causes the aqueous phase to gel over time.

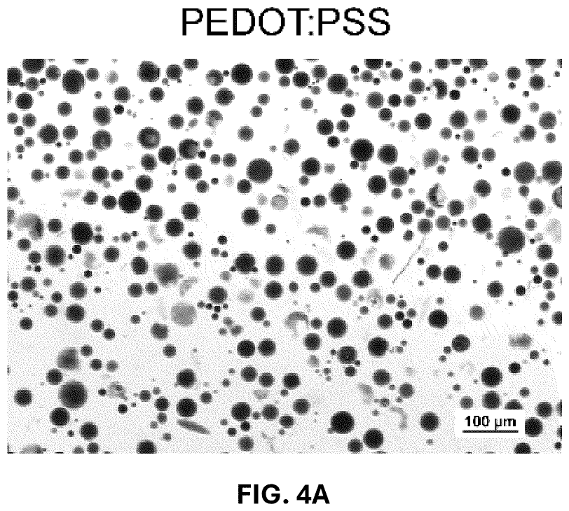

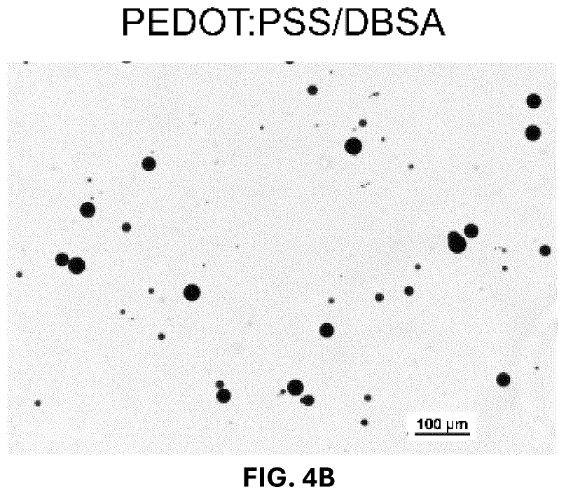

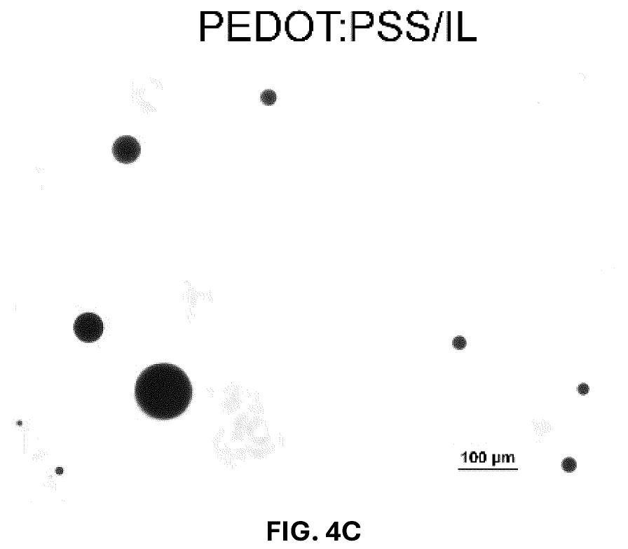

The mineral oil can be removed through various washes with 1×PBS. Essentially centrifugation is used to pack all of the microgels down to the bottom of a tube. As much of the mineral oil that can be removed without disturbing the particles is removed. 1×PBS is added to the tube and the tube is centrifuged again. Due to density differences, the microgels will again be at the bottom of the tube surrounded by 1×PBS. The least dense portion of the tube, the mineral oil, will be forced to the top allowing for even further removal of the oil. This process is repeated several times to ensure no mineral oil is left, and the microgels are left suspended in 1×PBS as seen in FIG. 3A. This process has also been completed to form microgels with pure PEDOT:PSS and PEDOT:PSS with a different gelation agent, dodecyl benzene sulfonic acid (DBSA) (FIGS. 4A and 4B, respectively).

Size ranges have been successfully isolated from the overall population (FIGS. 5A and 5B). This is done by using filters with various pore sizes. For example, the top image is the population of PEDOT:PSS/IL microgels before size selection. The bottom is an image of PEDOT:PSS/IL microgels that fell between 10 and 60 micrometers and were isolated from the original population in the top image using filters with pore sizes of 10 and 60 micrometers. The graph on the right shows how the distribution of the microgel diameter was narrowed from between 10 and 200 micrometers to between 10 and 60 micrometers.

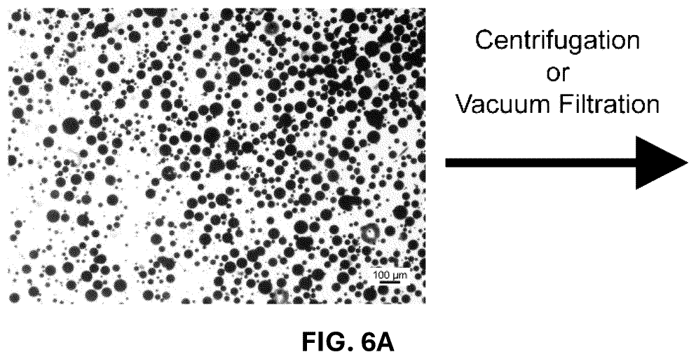

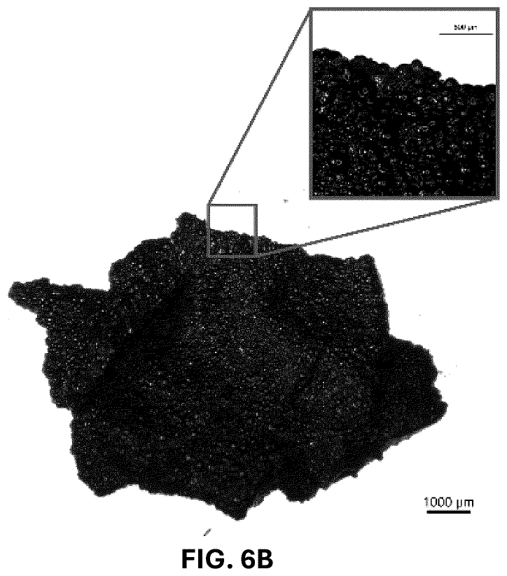

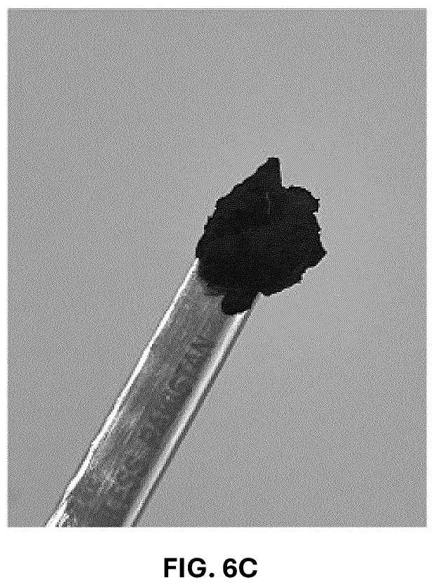

The PEDOT:PSS microgels are “jammed” to create a granular hydrogel. Essentially, centrifugation or vacuum filtration is used to remove as much aqueous phase surrounding the microgels as possible. As seen in FIG. 6A, the microgel suspension consists of the individual microgels suspended in a large amount of aqueous phase. Centrifugation or vacuum filtration is used to pack the microgels down to the bottom portion of the tube. FIG. 7 is a fluorescent image of jammed microgels acting as a granular hydrogel. The green portion of the image is the porous space filled with a fluorescent aqueous phase. The round black circles are the PEDOT:PSS/IL microgels jammed tightly together.

The porous space is used to define the void fraction of the material. As seen in FIG. 7, the void fraction is the volume of the porous space divided by the volume of the entire sample. It was discovered that centrifugal force impacts the amount of aqueous phase left behind in the granular material. The lower the centrifugal force, the more aqueous phase is left between particles. This contributes to a higher void fraction. The void fraction has been shown in previous research to impact the behaviors of cells encapsulated within the material. It was hypothesized it would also impact the printability of the material and the conductivity of the material.

When jammed, the granular hydrogel can be extruded as a strand and printed in various configurations. By way of non-limiting example, rheological testing was used to confirm that the granular hydrogel is shear thinning and self-healing. As illustrated in FIG. 9A, the material's viscosity decreases in response to increases in shear rate, confirming that the granular hydrogel material is shear-thinning.

As illustrated in FIG. 9B, the storage modulus and loss modulus decrease with the magnitude of oscillation strain at different rates, resulting in a crossover between the storage modulus and loss modulus, and indicating that the granular hydrogel material is strain-yielding. The point where the storage modulus (G′) crosses over with the loss modulus (G″) marks the transition from a solid-like state to a liquid-like state where the material can flow. The crossover point of the PEDOT:PSS granular hydrogel is 4.36% strain.

FIG. 9C demonstrates the self-healing properties of the granular hydrogel materials. The original values of the storage and loss moduli observed at a low shear strain (0.5%) are restored following the application of a high shear strain (500%, shaded). The average storage modulus (G′) at the low strain (0.5%) is 5308.01 Pa and the average loss modulus (G″) is 1193.89 Pa. 75% of that average storage modulus is immediately recovered after the application and removal of high strain; 92% of the initial storage and loss moduli values were recovered after five minutes.

The strain-yielding and self-healing properties of the granular hydrogel material enhances the material's ability to be formed into various structures using various fabrication techniques including, but not limited to, extrusion and 3D printing. When the granular hydrogel material undergoes shear force, for example shear forces generated within from 3D printing nozzles, the material begins to flow. When the shear force is removed, the granular hydrogel material returns to solid-like behavior. These attributes contribute to the material's ability to be extruded in various configurations and maintain those configurations after extrusion.

Without being limited to any particular theory, the void fraction of the granular hydrogel is thought to influence the extrusion properties of the hydrogel material including, but not limited to, minimum extrusion pressure and associated dimensions and precision of extruded filaments and other structures. If lower centrifugal forces are used to pack the granular hydrogel in a “jammed” form, it is assumed that the resulting larger void fraction (more porous space) would lead to a lower minimum extrusion pressure. Because the granular hydrogel materials with larger void fractions are more easily extruded with lower minimum extrusion pressures, filaments with greater widths are formed as compared to granular hydrogel materials with a lower void spaces extruded at similar conditions.

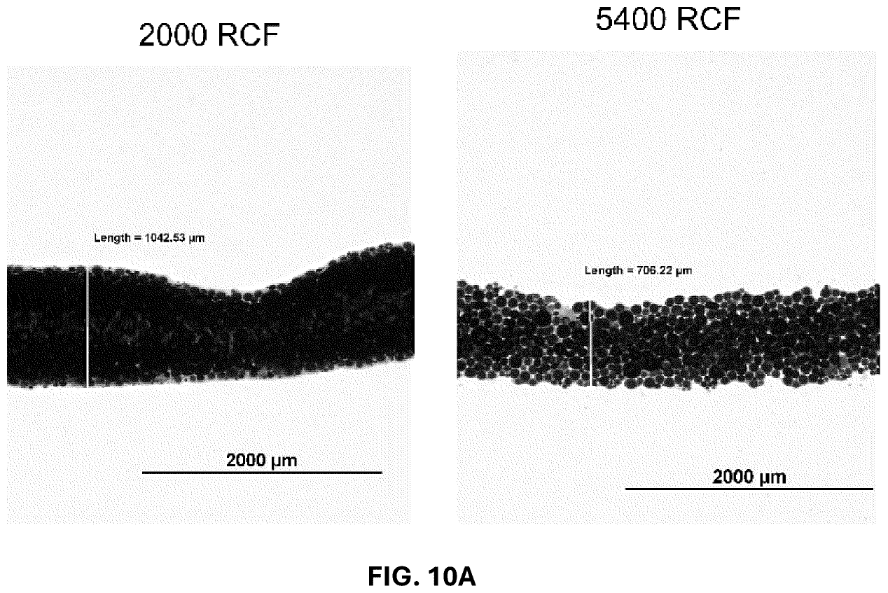

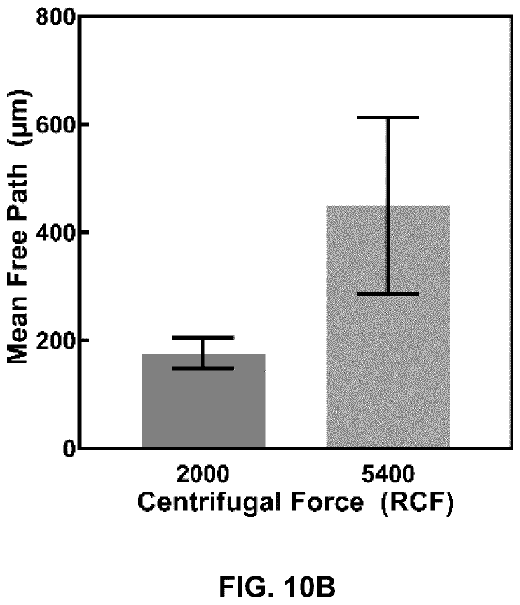

This association of larger void space with higher diameters of extruded filaments is demonstrated in the images of FIG. 10A that compare 3D printed filaments of the granular hydrogel material formed using lower (left) and higher (right) centrifugal forces. The right image of FIG. 10A shows a printed filament from our granular hydrogel material jammed with a higher centrifugal force. The granular hydrogel formed using a lower centrifugal force (left image), characterized by more aqueous phase within the material (a higher void fraction) formed filaments with greater widths than the filaments generated from material jammed at a higher centrifugal force (right image). Strands extruded through a 20-gauge nozzle (580 micrometer inner diameter) after packing at 2000 RCF possessed an average strand width of 872.368 micrometers and a standard deviation of 106.609 micrometers. Strands extruded after packing at a higher centrifugal force of 5400 RCF, which possess an average strand width of 729.236 micrometers and a standard deviation of 61.49023

In various aspects, the void fraction of the granular hydrogels is inversely proportional to their conductivity, i.e. granular hydrogels with lower void fractions have higher conductivities relative to granular hydrogels with higher void fractions. Without being limited to any particular theory, the increased conductivity of granular hydrogels formed using higher centrifugal forces (lower void fraction) is likely due to an increased frequency of microparticle-microparticle contact and a decrease in the average distance between microparticles within the granular hydrogel material.

By way of non-limiting example, the mean conductivities of various granular hydrogel materials formed from PEDOT:PSS/ionic liquid microparticles made with 10 mg/ml of ionic liquid and compacted at different centrifugal forces were measured and compared, as summarized in Table 1 below. Referring to Table 1, the measured conductivity of granular hydrogel material increased as the centrifugal forces used to pack the hydrogels increased. Consequently, the data of Table 1 demonstrate that granular hydrogel materials with lower void fractions (i.e. formed at higher centrifugal forces) have higher conductivities relative to granular hydrogel materials with higher void fractions (i.e. formed at lower centrifugal forces).

TABLE 1
Measured Conductivities of Granular Hydrogel Materials
Packed Using Different Centrifugal Forces

| Centrifugal Force (RCF) | Conductivity (Siemens/meter) |
|---|---|
| 800 | 1.27 |
| 3100 | 16.24 |
| 5400 | 50.96 |

In some aspects, the average distance between microparticles in the granular hydrogel material is quantified using mean free path, defined herein, refers to the length of free paths or paths of connected microparticles across the hydrogel material samples. Mean free path is determined by measuring paths of microparticles from one portion of the granular hydrogel to another until the path was interrupted by a gap or the path changed more than 90 degrees in direction. FIG. 10C is a bar graph comparing the mean free path of granular hydrogel materials formed using lower (2000 RCF) and higher (5400 RCF) centrifugal forces. The average mean free path of the material formed at a centrifugal force of 2000 RCF was 194.153 micrometers, and the average mean free path of the material formed at a centrifugal force of 5400 RCF was 440.118 micrometers.

Because the granular hydrogel materials are held together by non-specific forces, the shapes and configurations can be disturbed if any mechanical forces are introduced. However, one or more of at least several properties of the granular hydrogel material may mitigate the impact of disturbance by mechanical forces. By way of non-limiting example, the granular hydrogel material can be moved using a spatula and the addition of water will resuspend the granular hydrogels into microgels suspended in an aqueous phase, as illustrated in FIG. 6C.

By way of another non-limiting example, a collagen hydrogel overlay can be applied over the top of the granular hydrogel to keep mechanical forces from disrupting the granular hydrogel state as illustrated in FIG. 11. As illustrated in FIG. 11, the overlay does not penetrate or disrupt the granular hydrogel state. The collagen hydrogel overlay was applied over the top of the granular hydrogel to keep mechanical forces from disrupting the granular hydrogel state in a 35 mm petri dish used. Addition of water to the granular hydrogel with collagen overlay did not disperse particles and the granular hydrogel remained intact. The overlay interacted so little with the granular hydrogel that the overlay could be removed and the microgels resuspended in water.

It was also shown that the centrifugal force impacts the granular hydrogel's electronic conductivity (FIG. 10B). Lower centrifugal forces leave more aqueous phase in the granular hydrogel. As such, the surface area of microgel-microgel contact is much lower resulting in lower conductivity. Higher centrifugal forces remove more aqueous phase and the individual microgels are much more densely packed together. This results in greater microgel-microgel contact surface area and higher conductivity. The low centrifugal force (800 RCF) resulted in a conductivity of 1.268 S/m. The medium centrifugal force (3100 RCF) resulted in a conductivity of 16.24 S/m. Finally, the high centrifugal force (5400 RCF) resulted in a conductivity of 52.00 S/m.

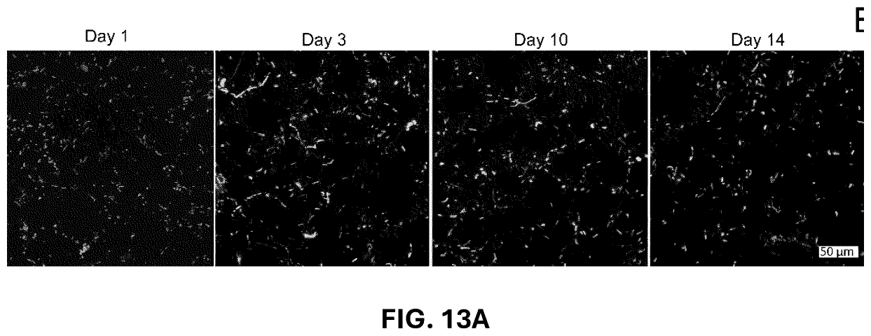

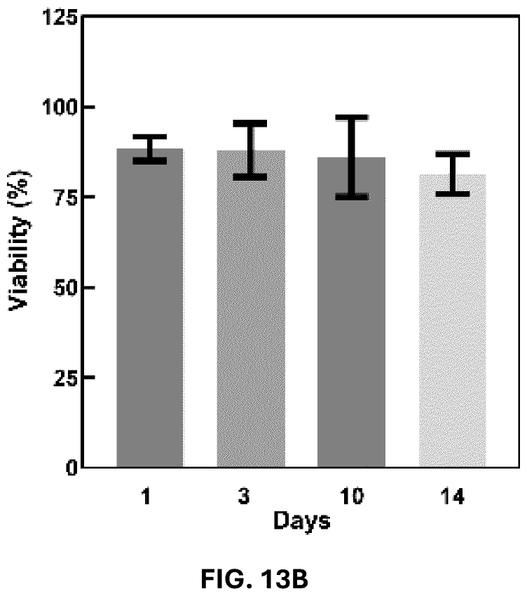

Normal human dermal fibroblasts were encapsulated within the microgels, and the cells were cultured for 14 days to prove the material was cytocompatible (FIGS. 13A and 13B). Cell viability was evaluated using a LIVE/DEAD assay. Green cells indicate they are alive while red cells indicate they are dead. The images above show that over 14 days, live cells are maintained and clearly in greater numbers than the dead cells. The result of this experiment indicates that that the granular hydrogel is cytocompatible.

One of the applications of the granular hydrogel bioink is the creation of 3D in vitro environments (FIG. 14). This will be done by the encapsulation of cells within the material and 3D printing of the now bioink onto an electrode. Current state-of-the-art set-ups involve depositing cells by hand on top of the conducting hydrogel. The process shown in the image of FIG. 14 is anticipated to generate a 3D set-up as shown in FIG. 14B. In this setup, cells will be surrounded by conducting polymer. It is hypothesized the increased cell-conducting granular hydrogel surface area will result in improved signal transduction and generate signals with greater amplitude and less noise.

The use of 3D bioprinting will also allow for the creation of 3D in vitro environments with spatially defined cell populations. Current state-of-the-art setups involve depositing cells by hand on top of multi-electrode arrays. This can lead to multiple cells being in contact with one electrode while others are left bare. 3D bioprinting would allow for precise depositions of different cell populations within the granular hydrogel bioink on selected electrodes (FIG. 14B). This will allow for the study of various biological activity across different cell types at the same time.

What is claimed is:

1. A conductive granular hydrogel composition for biomedical applications, the composition comprising a plurality of conductive hydrogel microparticles defining a plurality of interconnected micropores, each hydrogel particle comprising a conductive poly(3,4-ethylene-dioxythiophene):polystyrene sulfonate (PEDOT:PSS) composite polymer, wherein the composition comprises a conductivity of about 50-140 S/m.

2. The composition of claim 1 wherein the composition further comprises a void fraction value ranging from about 0.1 to about 0.95.

3. The composition of claim 1, wherein the composition is further configured to encapsulate living cells.

4. The composition of claim 1, wherein the composition is configured to be administered by a method selected from injection, external application, and any combination thereof.

5. The composition of claim 1, wherein the conductive hydrogel particles further comprise a gelation agent, the gelation agent comprising an ionic liquid.

6. The composition of claim 1, wherein the conductive hydrogel particles further comprise a plurality of living cells, a plurality of microparticles comprising one or more therapeutics, and any combination thereof.

7. A bioelectronic device, the device comprising a conductive granular hydrogel composition, the composition comprising a plurality of conductive hydrogel particles defining a plurality of interconnected micropores, each hydrogel particle comprising a conductive poly(3,4-ethylene-dioxythiophene):polystyrene sulfonate (PEDOT:PSS) composite polymer, wherein the composition comprises a conductivity of about 50-140 S/m and a void fraction value ranging from about 0.1 to about 0.95.

8. The device of claim 7, wherein the bioelectric device comprises at least one wearable electrode comprising the conductive granular conducting hydrogel composition applied over an external surface of an organism.

9. The device of claim 7, wherein the bioelectric device comprises at least one conductive filament comprising the conductive granular hydrogel composition, wherein the conductive filament is formed by extruding the composition using a needle of syringe or a nozzle of a printing device.

10. The device of claim 7, wherein the bioelectric device comprises a 3D in vitro cell environment comprising a cell culture medium, and a plurality of cells embedded within the conductive granular hydrogel composition.

11. The device of claim 10, wherein the cell culture medium further comprises a stabilizing agent selected from an ionic compound comprising NaCl, and amino acid comprising tryptophan, phenylalanine, alanine, any salt thereof, and any combination thereof, wherein the stabilizing agent maintains the structure of the conductive granular hydrogel composition.

12. The device of claim 7, wherein the device comprises a bioink, the bioink comprising a plurality of living cells embedded in the conductive granular hydrogel composition, wherein the bioink is configured to be extruded through a nozzle of a printing device.

13. A method of fabricating a granular conducting hydrogel, the method comprising
a. forming an oil phase mixture by combining a mineral oil and a surfactant;
b. forming an aqueous phase solution comprising a poly(3,4-ethylene-dioxythiophene):polystyrene sulfonate (PEDOT:PSS) composite polymer;
c. rapidly stirring the oil phase mixture at a temperature of about 90° C.;
d. introducing the aqueous phase solution into the stirring oil phase mixture to form an emulsion comprising droplets of the aqueous phase solution stabilized within the oil phase mixture;
e. gelling the aqueous phase mixture within the droplets in the stirring oil phase mixture to form conductive PEDOT:PSS hydrogel microparticles suspended in the oil phase mixture;
f. separating the hydrogel microparticles from the oil phase mixture by rinsing with phosphate buffered saline (PBS); and
g. packing the separated hydrogel microparticles to a predetermined void fraction value to produce the conductive granular hydrogel composition, the conductive granular hydrogel comprising the hydrogel microparticles packed together in a jammed state, wherein the hydrogel microparticles define a plurality of interconnected micropores.

14. The method of claim 13, wherein reducing a spacing between the separated hydrogel microparticles further comprises centrifuging the separated hydrogel microparticles, subjecting the separated hydrogel microparticles to vacuum filtration, and any combination thereof.

15. The method of claim 14 wherein the centrifuging is conducted at a centrifugal force ranging from about 2000 RCF to about 6000 RCF.

16. The method of claim 14, wherein the void fraction value ranges from about 0.1 to about 0.95 when created using centrifugation, or from about 0.1 to 0.3 when created using vacuum filtration.

17. The method of claim 13, wherein the surfactant comprises Span-80, wherein the Span-80 stabilizes the emulsion.

18. The method of claim 13, wherein the aqueous phase mixture further comprises a gelation agent, the gelation agent comprising an ionic liquid.

19. The method of claim 13, further comprising filtering the hydrogel microparticles suspended in the oil phase mixture to select a monodisperse portion of the hydrogel microparticles with diameters between 10 μm and 60 μm.

20. The method of claim 13, wherein the further comprising post-treating the hydrogel microparticles to remove at least a portion of an insulating portion of the composite polymer comprising PSS, wherein the post-treated conducting hydrogel composition comprises a conductivity of about 50-140 S/m.
